(19) United States
(12) Patent Application Publication
(10) Pub. No.: US 2011/0125101 A1
(43) Pub. Date: May 26, 2011

(54) OTOLOGIC SYRINGE NEEDLE

(76) Inventor: Yoshitoshi Koyama, Tokyo (JP)

(21) Appl. No.: 13/054,454

(22) PCT Filed: Jul. 15, 2008
(86) PCT No.: PCT/JP2008/062756

§ 371 Date: Jan. 14, 2011

(51) Int. Cl. A61M 5/32

(52) U.S. Cl. 604/239

(57) ABSTRACT

[Problem to be Solved] To provide an otologic syringe needle for accurately injecting and dispersing a medical agent into an extremely small region such as a middle ear within an eardrum.

[Means for Solving Problem] An otologic syringe needle 10 for passing through an eardrum and injecting a medical agent into cartilage within a middle ear, wherein a length of the needle is in the range of 40 to 60 mm, a cutting angle R of a cutting part 12 is in the range of 10 to 16 degrees, and a diameter D of the needle has gauge of not less than 27. The above-mentioned constitution enables accurate injection and dispersion of a medical agent into a desired place of cartilage located in a middle ear within an eardrum. In more detail, since the needle is reduced in diameter and cutting angle compared with conventional needle, the damage of an eardrum generated when the needle is passed through the eardrum can be avoided as much as possible. Further, aches generated in general injection treatments including an injection treatment for child in an otologic treatment can be reduced.

### FIELD OF THE INVENTION

The present invention relates to a syringe needle, particularly an otologic (otolaryngological) syringe needle for passing through eardrum and injecting a medical agent into a middle ear.

### DESCRIPTION OF THE RELATED ART

A conventional syringe needle used in an otologic treatment has a length of needle of approx. 60 mm, a diameter of needle of approx. 23 as gauge and a cutting angle of a tip portion of the needle of 17 to 21 degrees. The “gauge” indicating a diameter of the syringe needle means “X” of 1 inch/“X”. One inch is 25.4 mm and therefore 23 gauges mean approx. 1.10 mm.

As an effective treatment in an otorhinology including a treatment of hypacusia, instead of a systemic administration treatment done by oral route, injection or infusion of medical agents such as steroid, a local administration treatment that medical agents such as steroid is directly injected into inner year is proposed. In the latter case, it is necessary that the syringe needle is passed through an eardrum. Further, in case multiple injections using the syringe needle is carried out for the treatment, the eardrum suffers from multiple passes of the syringe needle. Furthermore, since the injection is carried out by passing through the eardrum, the injection is performed under such condition that the place to inject the medical agent cannot be looked. In more detail, when a medical agent for injection is injected into a round window located between an inner ear and middle ear, the treatment comes to be extremely difficult.

Patent Document 1 discloses a syringe needle for accurately injecting into a narrow place (area) of human body in an otologic treatment. In the invention of the Patent Document 1, the object is to provide a process highly facilitating the injection of treatment material into a mucous membrane in a region located within narrow tuba or between gathers and a syringe needle for using the process, and the syringe needle has a structure that only a portion for inserting into a mucous membrane of a tip of a syringe needle is bent diagonally forward and a guide part is provided so as to be projected forward from a starting portion of the bent part.

In more detail, an insert part provided on a tip of a shaft part having a lumen (tube) and provided with another lumen connecting the shaft part to the former lumen is projected diagonally forward in the longitudinal direction of the shaft, and the tip of shaft part further has a guide part which is projected forward in the longitudinal direction of the shaft. Hence, the tip of the syringe needle has a biforked shape.

The structure of the tip restricts the depth inserted by the needle to a predetermined range whereby excessive or insufficient insertion can be prevented. Hence, in a region rendering treatment with directly looking the region difficult, the region including, for examples, narrow tubes of human body such as auditory tube (otosalpinx) and ureter and gathers such as middle nasal meatus, the injection of treatment material into the mucous membrane can be easily and accurately carried out in the predetermined depth without skilled performance.

### List of Patent Documents:

Patent Document 1: JP2005-211397 A

### DISCLOSURE OF THE INVENTION

### Problem to be Solved by the Invention

A conventional syringe needle used in an otologic treatment does not have a specific structure that is considered from the viewpoint of the needle passing through an eardrum, and therefore the needle having the diameter mentioned previously is required to pay attention to reduction of hearing ability because the eardrum suffers from occurrence of hole due to the multiple passes of the syringe needle. Further, there is a problem that general aches generated from an injection treatment for human body including child are reduced.

In addition, it is highly difficult to inject a medical agent into a narrow place (area) such as a round window through an eardrum, because the injection is necessarily carried out in a restricted area and thickness. Therefore the use of the general syringe needle requires high proficiency for preventing the adverse current of the medical agent and keeping accurate injection.

The technique described in Patent Document 1 enables the insertion in accurate depth of the syringe needle into a place to be injected. However, a technique for injecting and dispersing a medical agent into an extremely small area including a soft region such as cartilage within a middle ear, is desired.

Recently, it has been recognized that the technique for injecting a medical agent into an inner or middle ear is used not only in the treatment of the regions associated with ear but also suitably in the treatment of the other regions. Therefore, it is important to propose a new or improved syringe needle for injecting a medical agent into an inner or middle ear through an eardrum.

In view of the above-mentioned problems, the object of the present invention is to provide an otologic (otolaryngological) syringe needle for accurately injecting and dispersing a medical agent into an extremely small area such as a middle ear within an eardrum.

### Means for Solving Problem

The present invention described in claim 1 to achieve the object can be provided by an otologic syringe needle for passing through an eardrum and injecting a medical agent into cartilage within a middle ear, wherein

a length of the needle is in the range of 40 to 60 mm,

a cutting angle of a tip portion of the needle is in the range of 10 to 16 degrees, and

a diameter of the needle has gauge of not less than 27.

The above-mentioned constitution enables accurate injection and dispersion of a medical agent into a desired place (area) of cartilage located in a middle ear within an eardrum. In more detail, since the needle is reduced in diameter and cutting angle compared with a conventional needle, the damage of an eardrum generated when the needle is passed through the eardrum can be avoided as much as possible. Further, aches generated in general injection treatments including an injection treatment for child in an otologic treatment can be reduced. Furthermore, the reduced cutting angle increases the opening area of the tip portion of the needle, and therefore brings about increase of area of the opening area in contact with a place to be injected. Hence, an area for injection of a medical agent is increased, and therefore the medical agent can be accurately dispersed in the injected place.

In the preferred embodiment of the otologic syringe needle described in claim 2, which has the constitution of claim 1, the cutting part in the tip portion has a length of not more than 2 mm in an axial direction of the needle. Thereby, the area of the cutting part has a length which fits a cartilage covering a round window in an inner ear where the cutting part is frequently applied, and hence it can be prevented that the opening part of the tip portion runs off the edge of the applied area to cause the medical agent to leak.

In the preferred embodiment of the otologic syringe needle described in claim 3, which has the constitution of claim 1, at least one small hole is formed on the cutting part, which corresponds to a remaining portion constituting an opening area in the tip portion. Thereby, the medical agent is injected and dispersed from not only the opening area of the cutting part but also the hole(s) of the tip potion, and therefore the injection of the medical agent in various directions is ensured whereby the dispersibility of the medical agent can be enhanced.

In the preferred embodiment of the otologic syringe needle described in claim 4, which has the constitution of claim 1, at least one slit having a predetermined length is formed on the cutting part, which corresponds to a remaining portion constituting an opening area in the tip portion. Thereby, the medical agent is injected and dispersed from not only the opening area of the cutting part but also the slit(s) of the tip potion, and therefore the injection of the medical agent in various directions is ensured whereby the dispersibility of the medical agent can be enhanced.

In the preferred embodiment of the otologic syringe needle described in claim 5, which has the constitution of claim 4, a width of the slit is gradually increased with moving to a pointed tip of the needle. Thereby, the gradually widened slit is provided on a different side from that of the conventional opening, and therefore the injection area of the medical agent can be increased.

In the preferred embodiment of the otologic syringe needle described scribed in claim 6, which has the constitution of claim 4 or 5, a plurality of slits are formed as the slit. Thereby, the dispersibility of the medical agent can be further enhanced compared with the embodiment of claim 4 or 5.

In the preferred embodiment of the otologic syringe needle described in claim 7, which has the constitution of any of claims 1 to 6, a longitudinal sectional shape in a needle axial direction of the cutting part in the tip portion is in the form of circular arc which forms convex on the cutting surface side. Thereby, in case the length in a needle axial direction of the cutting part is the same between the above-mentioned needle having the convex shape and the needle having no convex shape, the above-mentioned needle enables the increase of the area of the opening area in contact with an area to be injected. Further, in case the opening area of the cutting part is the same between the above-mentioned needle having the convex shape and the needle having no convex shape, the strength of the cutting part of the above-mentioned needle having the convex shape on the surface side is increased. Hence the above-mentioned needle shows the significance effects as mentioned above when both the diameter and cutting angle of the cutting part are reduced.

### Effect of the Invention

As mentioned previously, the use of the otologic syringe needle of the present invention brings about the reduction of damage of an eardrum and the enhancement of injection accuracy of a medical agent, in case the syringe needle is passed through the eardrum and the medical agent in the syringe is injected into a narrow cartilage part such as round window of an inner ear located within the eardrum. Further aches generated in an injection treatment for child in an otologic treatment can be reduced, and aches generated in a general injection treatment can be also reduced. Thus, a treatment rejecting a medical agent through an inner ear can be widely carried out.

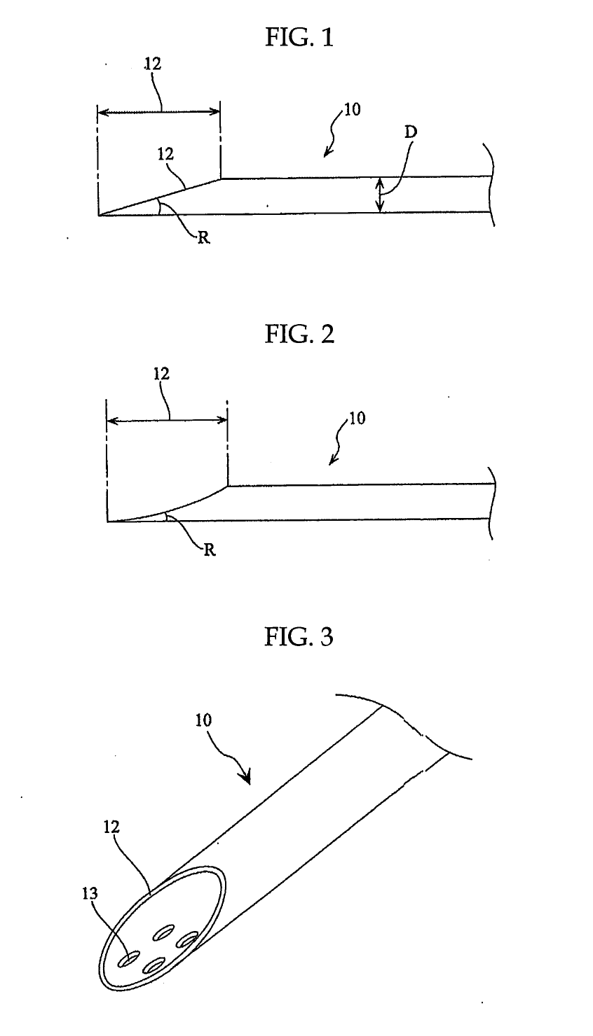

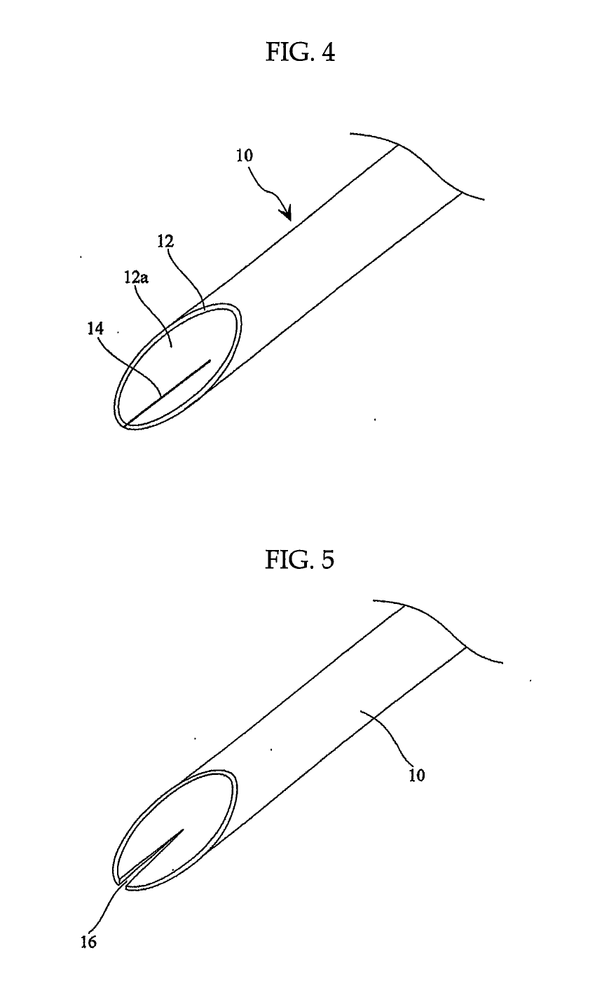

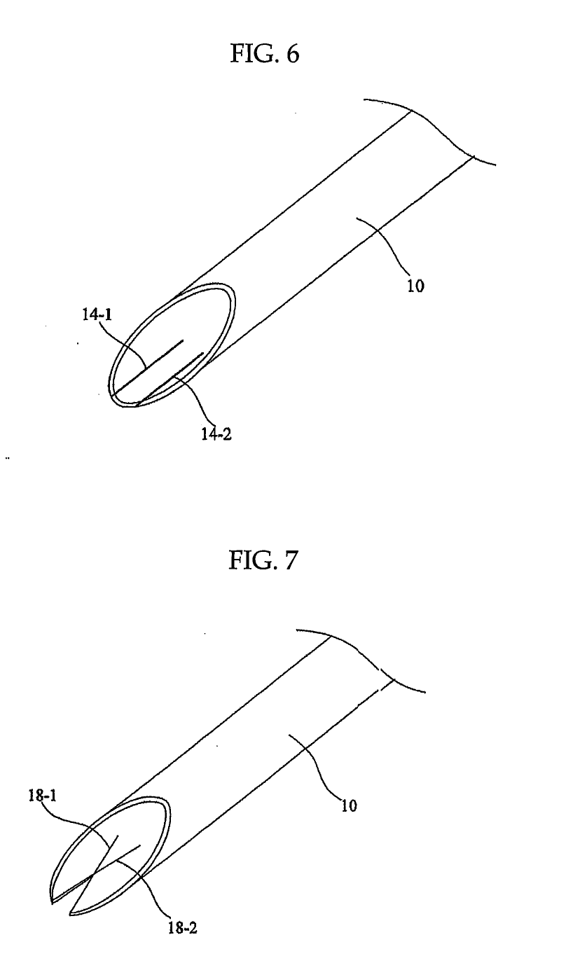

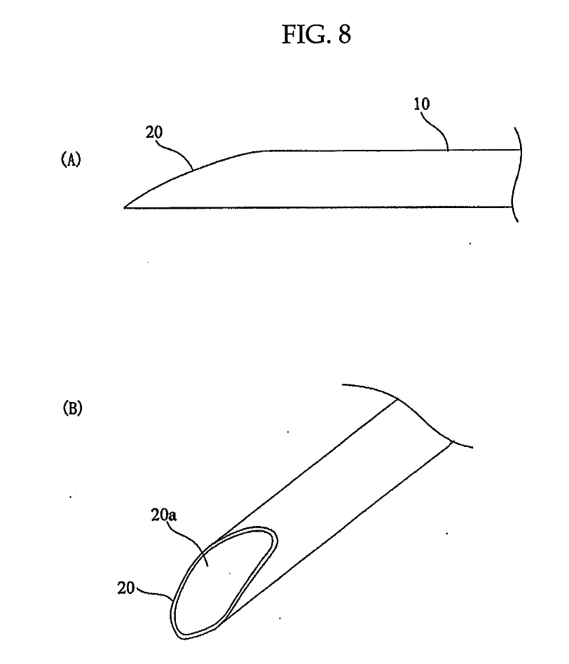

### BRIEF DESCRIPTION OF THE DRAWINGS

[FIG. 1] FIG. 1 is a view for explaining an outline constitution of the tip portion of the otologic syringe needle according to an embodiment of the present invention.

[FIG. 2] FIG. 2 is a view for explaining an outline constitution of the tip portion of the otologic syringe needle according to another embodiment of the present invention, the cutting line being nonlinear.

[FIG. 3] FIG. 3 is a view for explaining an outline constitution of the tip portion of the otologic syringe needle according to another embodiment of the present invention, small holes being formed on the cutting part (i.e., remaining part) constituting an opening area in the tip portion.

[FIG. 4] FIG. 4 is a view for explaining an outline constitution of the tip portion of the otologic syringe needle according to another embodiment of the present invention, a slit being formed on the cutting part (i.e., remaining part).

[FIG. 5] FIG. 5 is a view for explaining an outline constitution of the tip portion of the otologic syringe needle according to another embodiment of the present invention, a slit being formed on the cutting part (i.e., remaining part).

[FIG. 6] FIG. 6 is a view for explaining an outline constitution of the tip portion of the otologic syringe needle according to another embodiment of the present invention, two slits being formed on the cutting part (i.e., remaining part).

[FIG. 7] FIG. 7 is a view for explaining an outline constitution of the tip portion of the otologic syringe needle according to another embodiment of the present invention, crossed two slits being formed on the cutting part (i.e., remaining part).

[FIG. 8] FIGS. 8(A) and (B) are a view for explaining an outline constitution of the tip portion of the otologic syringe needle according to another embodiment of the present invention, the cutting part in the tip portion being in the form of circular arc which is bunchy on the cutting surface side.

### EXPLANATION OF REFERENCE NUMBER

10 Syringe needle

12 Cutting part

12a Cutting opening area

13 Small hole

14, 16, 18 Slit

20 Circular arc shaped cutting part

### DESCRIPTION OF PREFERRED EMBODIMENTS

The preferred embodiments are explained below with reference to the drawings. FIG. 1 is a view for explaining an outline constitution of the tip portion of the otologic syringe needle according to an embodiment of the present invention. A syringe needle 10 of the present invention has such length of needle that can be injected from the outside of an ear to the inside of the ear, and the length is generally configured in the range of 40 to 60 mm, preferably 45 to 55 mm. Further the diameter D of needle is generally adjusted to have 29 gauges (approx. 0.87 mm). Furthermore the tip portion of the syringe needle 10 is cut and the cutting angle R of the cutting part 12 is set to be 16 degrees.

The syringe needle is reduced in diameter and cutting angle compared with a conventional needle as mentioned above, and therefore the damage of an eardrum generated when the needle is passed through the eardrum can be avoided as much as possible. Further, aches generated in general injection treatments including an injection treatment for child in an otologic treatment can be reduced.

In case a medical agent such as steroid is injected by using a syringe into an area of a place to be injected such as a soft region adjacent to a round window of an inner ear, which is narrow, it is required to allow the cutting part of the needle to run off the edge of the place (region) to be injected in order to accurately inject the medical agent. For instance, in case the medical agent is injected into the region adjacent to the round window by using a syringe, the cutting part 12 should have generally a length of not more than 2 mm in an axial direction of the needle. In this case, it is also possible to increase the gauge number of the needle diameter (reduction of the diameter), or to set the cutting angle R to 16 degrees and incline upward and steeply the cutting line in the nonlinear form whereby the length of the cutting part 12 is set to 2 mm without change of the gauge of the needle, as shown in FIG. 2, in order to obtain a predetermined length of the pointed tip of the needle 10.

Further, the reduced cutting angle increases the opening area of the tip portion of the needle, and therefore brings about increase of area of the opening area in contact with a place to be injected. Hence, an area for injection of a medical agent is increased, and therefore the medical agent can be accurately dispersed in the injected place.

FIG. 3 shows another embodiment of the present invention. A syringe needle 10 of the embodiment has small holes 13 on the cutting part (i.e., remaining part) 12 in the tip portion in addition to the features of FIG. 1. This constitution not only increases the contact area of the cutting part but also enhance the dispersibility of the medical agent. In more detail, the medical agent is injected and dispersed from not only the opening area 12a of the cutting part but also the holes 13 of the tip potion, and therefore the injection of the medical agent is ensured in various directions on the upper and lower sides of the tip portion of syringe needle 10 whereby the dispersibility of the medical agent can be enhanced.

FIG. 4 shows another embodiment of the present invention, and in the embodiment, a slit 14 is formed on the cutting part 12 (i.e., remaining part) constituting an opening area in the tip portion of the syringe needle 10. Moreover, in the another embodiment of FIG. 5, also a slit 16 is formed on the cutting part 12 (i.e., remaining part) constituting an opening area in the tip portion of the syringe needle 10 and further a width of the slit is gradually increased with moving to a pointed tip of the needle. Thereby, another opening area formed on a different side from that of the conventional opening area 12a is further widened, and hence the increase of the injection area and the enhancement of the dispersion of the medical agent are ensured in addition to the above-mentioned effect. Further, it is possible to form a plurality of slits instead of the slit 14 or 16.

FIG. 6 shows another embodiment of the present invention, and in the embodiment, two slits 14-1, 14-2 are formed on the cutting part 12 (i.e., remaining part) constituting an opening area in the tip portion of the syringe needle 10. Thereby, the increase of the injection area and the enhancement of the dispersion of the medical agent are ensured as in the above-mentioned embodiment. The number of the slit is not restricted to two, and it is possible to form three or more slits on the tip portion of the syringe needle 10 unless the tip portion is reduced in strength. Further, the width and shape of the slit can be variously changed. For example, it is possible to adopt the shape that a width of the slit is gradually increased with moving to a pointed tip of the needle as shown in FIG. 5.

FIG. 7 shows another embodiment of the present invention, and in the embodiment, two slits 18-1, 18-2 are formed on the cutting part 12 (i.e., remaining part) constituting an opening area in the tip portion of the syringe needle 10 such that the two slits are crossed each other. Thereby the tip of the needle is divided into two directions, and further the two slits 18-1, 18-2 are formed continuously to connect to the missing part of the tip. Thereby, the increase of the injection area and the enhancement of the dispersion of the medical agent are ensured as in the above-mentioned embodiment.

FIGS. 8(A) and (B) show another embodiment of the present invention, and FIG. 8(A) is a side view and FIG. 8(B) is a perspective view. As shown in FIG. 8, a longitudinal sectional shape in a needle axial direction of the cutting part in the tip portion of the syringe needle 10 is formed in the form of circular arc which is convex on the cutting surface side. The conditions such as the cutting angle of the cutting part in the form of circular arc are the same as in the embodiments described above. As shown in FIG. 8, the circular arc shaped cutting part 20 has a little rounded convex shape, whereby in case the length in a needle axial direction of the cutting part is the same between the above-mentioned needle having the convex shape and the needle having no convex shape, the above-mentioned needle enables the increase of the area of the opening area 20a in contact with an area to be injected. Further, in case the opening area of the cutting part is the same between the above-mentioned needle having the convex shape and the needle having no convex shape, the above-mentioned needle has the rounded convex shape on the surface side and therefore the strength of the tip of the cutting part is also increased.

Here, it is possible to apply the technique of the circular arc shaped cutting part 20 having the rounded shape to a needle having cutting angle of more than 16 degrees. Although the large cutting angle brings about reduction of the opening area of the tip portion, the reduction can be compensated by the circular arc shape of the cutting part.

The present invention is not restricted by the embodiments mentioned above, and it is possible to change the invention to various embodiments as long as the embodiments are within the scope of the gist of the present invention. For example, the length of the slit formed on the cutting part (remaining part of opening area) can be varied unless the length runs off the edge of the place (area) to be injected.

1. An otologic syringe needle for passing through an eardrum and injecting a medical agent into cartilage within a middle ear, wherein
a length of the needle is in the range of 40 to 60 mm,
a cutting angle of a tip portion of the needle is in the range of 10 to 16 degrees, and
a diameter of the needle has gauge of not less than 27.

2. The otologic syringe needle as defined in claim 1, wherein the cutting part in the tip portion has a length of not more than 2 mm in an axial direction of the needle.

3. The otologic syringe needle as defined in claim 1, wherein at least one small hole is formed on the cutting part.

4. The otologic syringe needle as defined in claim 1, wherein at least one slit having a predetermined length is formed on the cutting part.

5. The otologic syringe needle as defined in claim 4, wherein a width of the slit is gradually increased with moving to a pointed tip of the needle.

6. The otologic syringe needle as defined in claim 4, wherein a plurality of slits are formed as the slit.

7. The otologic syringe needle as defined in claim 1, wherein a longitudinal sectional shape in a needle axial direction of the cutting part in the tip portion is in the form of circular arc which forms convex on the cutting surface side.
